(19) United States
(12) Patent Application Publication
(10) Pub. No.: US 2023/0166094 A1
(43) Pub. Date: Jun. 1, 2023

(54) PRN ADAPTER

(71) Applicant: Becton, Dickinson and Company, Franklin Lakes, NJ (US)

(72) Inventor: Linda Kunardi, Serangoon (SG)

(21) Appl. No.: 17/920,678

(22) PCT Filed: Apr. 19, 2021
(86) PCT No.: PCT/US2021/027899

§ 371 Date: Oct. 21, 2022

(30) Foreign Application Priority Data
Apr. 24, 2020 (CN) 202020642967.6

(51) Int. Cl. A61M 39/10

(52) U.S. Cl. CPC A61M 39/10; A61M 2039/1072

(57) ABSTRACT

A PRN adapter can be formed of a casing and an elastomeric member. The elastomeric member can form a septum and a male luer taper. By forming the PRN adapter of only two components, the manufacturing process can be simplified. The casing may form a hollow interior having a proximal opening and a distal opening. The septum of the elastomeric member may be positioned at the proximal opening of the hollow interior, and the male luer taper may extend to the distal opening of the hollow interior.

### BACKGROUND

Intravenous (IV) catheter devices are commonly used for a variety of infusion therapies. For example, an IV catheter device may be used for infusing fluids, such as normal saline solution, various medicaments, and total parenteral nutrition, into a patient. IV catheter devices may also be used for withdrawing blood from the patient.

A common type of IV catheter device is an over-the-needle peripheral intravenous (“IV”) catheter (“PIVC”). As its name implies, the over-the-needle catheter may be mounted over a needle having a sharp distal tip. The catheter and the needle may be assembled so that the distal tip of the needle extends beyond the distal tip of the catheter with the bevel of the needle facing up away from skin of the patient. The catheter and needle are generally inserted at a shallow angle through the skin into the vasculature of the patient.

IV catheter devices typically provide an access port by which other devices may obtain access to the catheter while the catheter is positioned in a patient’s vasculature. These other devices may be employed to perform various tasks such as obtaining a blood sample, injecting a fluid, performing a measurement, monitoring, etc. It is common to employ a “PRN adapter” to protect such access ports and to provide a valve at the access ports. The abbreviation “PRN” (from the Latin “pro re nata”) generally means “when necessary,” and represents that PRN adapters enable a clinician to access the patient’s vasculature “when necessary.”

FIG. 1 provides an example of a PIVC 100 with which a PRN adapter may be employed. PIVC 100 includes a catheter adapter 110 from which a catheter 111 extends, a needle adapter 120 from which a needle 121 extends and an extension set 130 having extension tubing 131 that extends from catheter adapter 110 to a Y-adapter 132 having two female luer lock ports 132a and 132b. A PRN adapter 140 is connected to port 132a and a vent plug 150 is connected to port 132b. FIG. 1 is therefore intended to represent that PRN adapters are commonly used on the ports of a Y-adapter or other connector of an extension set. Notably, PRN adapters may also be used on ports at other locations of a PIVC or with other IV catheter devices.

FIG. 2 provides an example of a common PRN adapter 240. PRN adapter 240 consists of three separate components: (1) a casing 241; (2) a septum 242; and (3) a shrink film 243. Casing 241, which is typically formed of a hard plastic, forms a male luer taper 241a that inserts into a female luer port (e.g., port 132a or 132b). Casing 241 will also typically be in the form of a male luer lock sleeve 241b to enable PRN adapter 240 to be threaded onto a female luer lock port. Septum 242, which is typically formed of an elastomeric material such as rubber or silicone, functions as a seal to prevent blood or fluid from escaping through PRN adapter 240 while also enabling a needle or cannula to be inserted therethrough to gain access to the PIVC or other IV catheter device to which PRN adapter 240 may be connected. Septum 242 may insert partially into casing 241. Shrink film 243 functions to secure septum 242 to casing 241. For example, during manufacturing of PRN adapter 240, shrink film 243 could be positioned around a portion of casing 241 and a portion of septum 242 and then heated to cause the shrink film to tighten around these portions.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to PRN adapters and to IV catheter devices on which PRN adapters may be employed. A PRN adapter can be formed of a casing and an elastomeric member. The elastomeric member can form a septum and a male luer taper. By forming the PRN adapter of only two components, the manufacturing process can be simplified.

In some embodiments, a PRN adapter can include a casing and an elastomeric member coupled to the casing. The elastomeric member forms a septum and a male luer taper. In some embodiments, the casing may form a hollow interior having a proximal opening and distal opening. In some embodiments, the septum of the elastomeric member may be positioned at the proximal opening of the hollow interior and the male luer taper may extend to the distal opening of the hollow interior. In some embodiments, the casing may include a rim that forms the proximal opening. In some embodiments, the septum may form a distally facing lip that overlaps a proximal face of the rim. In some embodiments, the elastomeric member may include a retaining member that is spaced from the septum by a neck. In some embodiments, a proximal face of the retaining member may abut a distal face of the rim. In some embodiments, the hollow interior may include threads. In some embodiments, the elastomeric member may form a lumen that extends from the septum through the male luer taper.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 illustrates an example of a common PIVC on which a PRN adapter could be used;

FIG. 2 illustrates an example of a common PRN adapter;

FIG. 3 illustrates an example of a PRN adapter that is configured in accordance with some embodiments of the present disclosure;

FIG. 3A is a cross-sectional view of the PRN adapter of FIG. 3;

FIG. 4 illustrates a septum member of the PRN adapter of FIG. 3; and

FIG. 4A is a cross-sectional view of the septum member of FIG. 4.

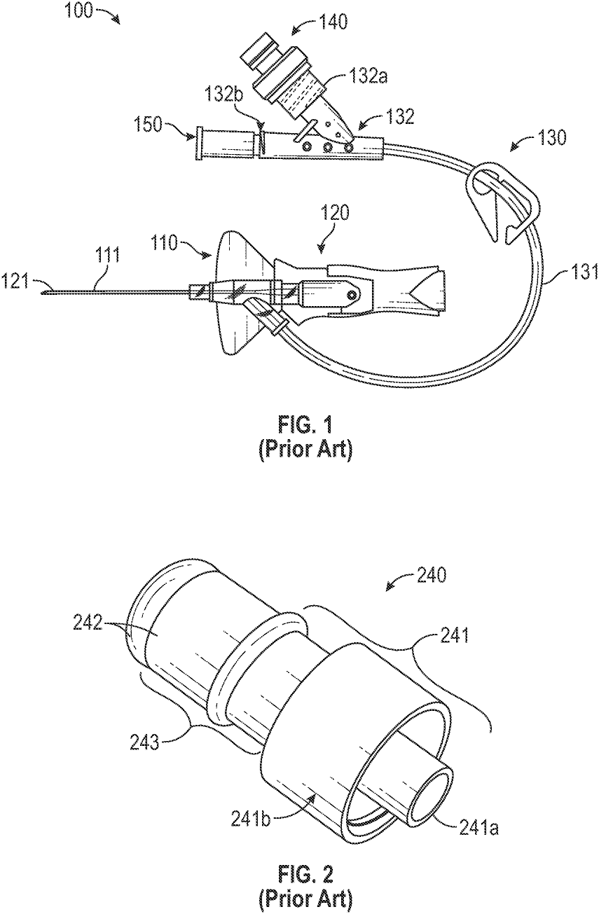

### DESCRIPTION OF EMBODIMENTS

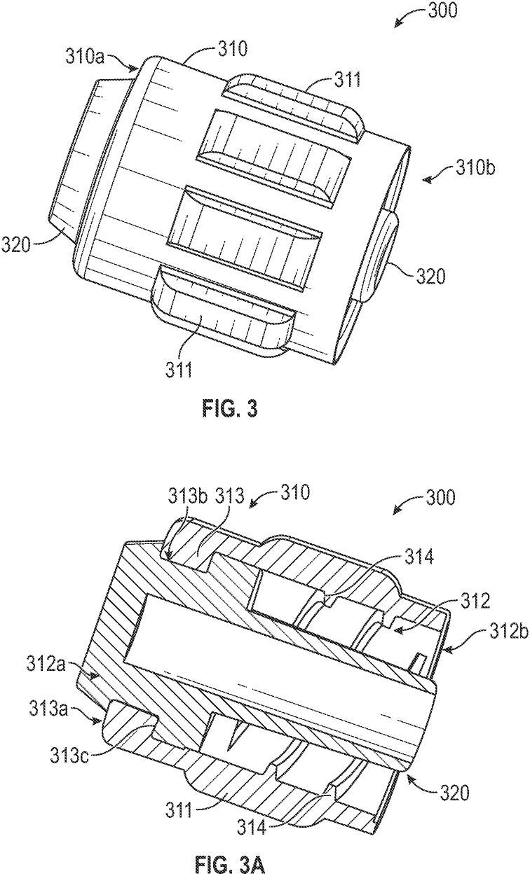

In the specification and the claims, the term “PRN adapter” should be construed as any adapter that may be part of or used on an IV catheter device to enable another device to gain access to the catheter of the IV catheter device while the catheter is positioned intravenously. FIG. 3 provides an example of a PRN adapter 300 that is configured in accordance with some embodiments of the present disclosure. FIG. 3A provides a cross-sectional view of PRN adapter 300.

PRN adapter 300 is formed of two components: (1) a casing 310; and (2) an elastomeric member 320. Casing 310 extends from a proximal end 310a to a distal end 3 10b. A hollow interior 312 of casing 310 forms a proximal opening 312a and a distal opening 312b. A rim 313 (or a reduced diameter region of hollow interior 312) forms proximal opening 312a. Rim 313 forms a proximal face 313a, an inward face 313b and a distal face 313c. Threads 314 are formed within hollow interior 312 and extend from distal opening 312b in a proximal direction. Ribs 311 may be formed around an exterior of casing 310 and may function to facilitate gripping of PRN adapter 300.

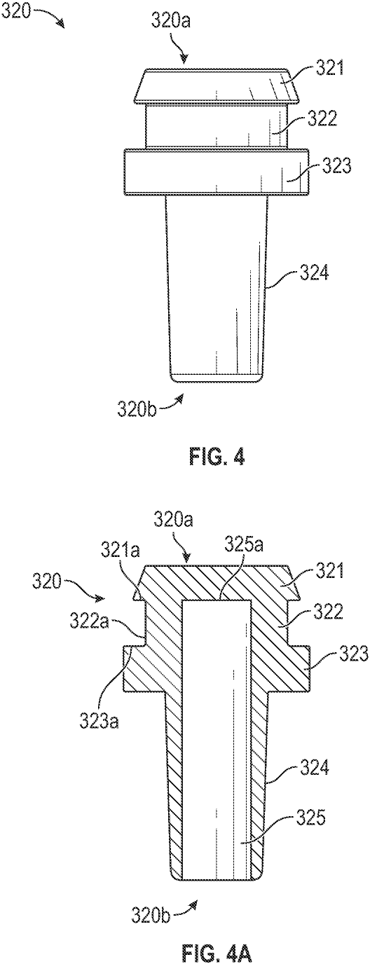

FIG. 4 provides an example of elastomeric member 320 in isolation, and FIG. 4A provides a cross-sectional view of elastomeric member 320 in isolation. As shown, elastomeric member 320 extends from a proximal end 320a to a distal end 320b. A septum 321 is formed at proximal end 320a and a male luer taper 324 is formed at distal end 320b. A neck 322 extends distally from septum 321, and a retaining member 323 extends distally from neck 322. Neck 322 comprises a reduced outer diameter region relative to an outer diameter of septum 321 and relative to an outer diameter of retaining member 323. Accordingly, a neck sidewall 322a is positioned between a distally-facing lip 321a formed on septum 321 and a proximal face 323a formed on retaining member 323.

Elastomeric member 320 also includes a lumen 325 that extends from a proximal end 325a adjacent septum 321 through distal end 320b of elastomeric member 320. In the depicted embodiment, proximal end 325a of lumen 325 aligns with lip 321a. However, in other embodiments, proximal end 325a may be offset relative to lip 321a such that the thickness of septum 321 is greater or lesser than what is shown in the figures.

As best seen in FIG. 3A, elastomeric member 320 is secured to casing 310 by positioning rim 313 between septum 321 and retaining member 323. In this position, lip 321a overlaps proximal face 313a of rim 313, neck sidewall 322a abuts inward face 313b of rim 313 and proximal face 323a of retaining member 323 abuts distal face 313c of rim 313. Accordingly, septum 321 is positioned proximal to rim 313 and male luer taper 324 extends distally beyond distal opening 312b. In other embodiments, however, male luer taper 324 may extend up to or short of distal opening 312b. The outer diameter of male luer taper 324 is less than the diameter of hollow interior 312 to enable a female luer lock port to insert between male luer taper 324 and casing 310 when male luer taper 324 is inserted into the female luer lock port.

The elastomeric property of elastomeric member 320 enables elastomeric member 320 to be inserted into and secured within casing 310. For example, elastomeric member 320 can be inserted through distal opening 312b until septum 321 passes through proximal opening 312a. Septum 321 can deform to enable it to pass through the smaller diameter of proximal opening 312a. Retaining member 323 can then prevent further distal movement of elastomeric member 320 relative to casing 310. With septum 321 positioned proximally beyond proximal opening 312a, lip 321a will overlap proximal face 313a to thereby prevent elastomeric member 320 from moving distally.

PRN adapter 300 can be connected to a female luer lock port of an IV catheter device by inserting male luer taper 324 into the port and turning PRN adapter 300 to tighten threads 314 against the corresponding tab(s) of the female luer lock port. As PRN adapter 300 is tightened onto the port, male luer taper 324 can form a fluid tight seal against the female luer lock port. To gain access to the catheter of the IV catheter device, a needle or cannula of a separate device can be inserted through septum 321 and into lumen 325. When the needle or cannula is subsequently removed, the elastomeric property of septum 321 will reform a seal and prevent any fluid from escaping lumen 325.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A PRN adapter comprising:
a casing; and
an elastomeric member coupled to the casing, the elastomeric member forming a septum and a male luer taper.

2. The PRN adapter of claim 1, wherein the casing forms a hollow interior having a proximal opening and a distal opening.

3. The PRN adapter of claim 2, wherein the septum of the elastomeric member is positioned at the proximal opening of the hollow interior and the male luer taper extends to the distal opening of the hollow interior.

4. The PRN adapter of claim 3, wherein the casing includes a rim that forms the proximal opening.

5. The PRN adapter of claim 4, wherein the septum forms a distally facing lip that overlaps a proximal face of the rim.

6. The PRN adapter of claim 5, wherein the elastomeric member includes a retaining member that is spaced from the septum by a neck.

7. The PRN adapter of claim 6, wherein a proximal face of the retaining member abuts a distal face of the rim.

8. The PRN adapter of claim 2, wherein the hollow interior includes threads.

9. The PRN adapter of claim 1, wherein the elastomeric member forms a lumen that extends from the septum through the male luer taper.

10. The PRN adapter of claim 1, wherein the casing forms a hollow interior having a proximal opening and a distal opening, the proximal opening being formed by a rim, the hollow interior including threads that extend from the distal opening; and
wherein the elastomeric member includes a retaining member that is spaced from the septum by a neck, the elastomeric member also forming a lumen that extends from the septum through the male luer taper, the septum of the elastomeric member being positioned at the proximal opening of the hollow interior and the male luer taper extending to the distal opening of the hollow interior, the septum forming a distally facing lip that overlaps a proximal face of the rim when a proximal face of the retaining member abuts a distal face of the rim.
